(19) United States
(12) Patent Application Publication
(10) Pub. No.: US 2015/0141849 A1
(43) Pub. Date: May 21, 2015

(54) METHOD AND DEVICE FOR NONINVASIVE BLOOD PRESSURE MEASUREMENT

(71) Applicant: Nicolay Verwaltung GmbH, Nagold (DE)

(72) Inventor: Edwin Muz, Reutlingen (DE)

(21) Appl. No.: 14/514,621

(22) Filed: Oct. 15, 2014

(30) Foreign Application Priority Data
Oct. 24, 2013 (DE) 10 2013 017 716.2

(51) Int. Cl. A61B 5/021; A61B 5/00; A61B 5/022

(52) U.S. Cl. CPC A61B 5/02116; A61B 5/022; A61B 5/0059
USPC 600/480; 600/492

(57) ABSTRACT

The invention relates to a method and a device for noninvasive blood pressure measurement by means of two pressure cuffs (10, 20) each having one sensor (6, 8), the output signal of which is a function of the blood pressure to be measured and of the cuff pressure, characterized in that, first in a reference phase, the cuff pressure of at least one pressure cuff is varied and the maximum output signals of the sensor occurring at the systolic blood pressure are detected at the different cuff pressures, the dependence of the maximum output signal from the sensor (6, 8), occurring at the systolic blood pressure, on the transmural pressure is determined, and the curve of the reference phase quotient is determined as a function of transmural pressure and saved, then, in a measurement phase during a pulse beat, two signal values are determined at two different cuff pressures by means of a control unit, and a measurement phase quotient is formed from these two signal values, and, by comparing the measurement phase quotient with the saved reference phase quotient, the blood pressure is determined.

The invention relates to a method and a device for a noninvasive measurement of the systolic and diastolic blood pressure values of a patient; these are suitable in particular for long-term monitoring of blood pressure and for recording a blood pressure curve.

Many methods for noninvasive measurement of blood pressure only allow measurements at longer intervals of time of 10 minutes, for example, and use empirical values for determining the systolic and diastolic blood pressure. The determination of the diastolic blood pressure value thereby is usually rather uncertain.

Indirect methods of measuring blood pressure are known, utilizing the transit time of the pulse wave from the heart to the measurement site, for example (see U.S. Pat. No. 4,869,262 and U.S. 2008/0039731) but neither of these is suitable for recording the pulse rate curve.

In addition, there are known methods, which construct a valid curve, the so-called arterial compliance curve dV/dPt, which is valid for the respective test subject on the basis of a theoretical approach and by measuring a few parameters and then use this for the measurement, see U.S. Pat. No. 4,846,181 and U.S. Pat. No. 5,423,322 which describes the change in the volume dV of the arteries beneath the blood pressure cuff in the measurement range as a function of the resulting pressure on the arterial wall, the so-called transmural pressure Pt which is defined as

Pt=Pi−Pm,

where Pi is the pressure inside the vessel and is the blood pressure, and Pm is the pressure in the cuff.

In addition, the so-called volume compensation method, which is based on an analysis of a signal depending only on the transmural pressure Pt, is also known; see U.S. Pat. No. 4,009,709, U.S. Pat. No. 4,846,189, U.S. Pat. no. 4,074,711, WO 2005/037097, EP 0 073 123 and EP 0 060 252. In this method, the cuff pressure is controlled, so that the signal

S=const·f(Pi−Pm)

is always constant and therefore Pm is always equal to Pi. The equipment complexity here is very high, and furthermore, there is a great burden on the patient because the arteries are temporarily closed off completely, so that a longer-term measurement is hardly feasible.

The present invention is thus based on the object of providing a method and a device for noninvasive blood pressure measurement, which will overcome the disadvantages of the prior art. In particular the burden on the patient is also to be reduced through continuous blood pressure measurement, if applicable.

This object is achieved by the method defined in claim 1 and by the device defined in the other independent claim. Particular embodiments of the invention are defined in the dependent claims.

In summary, the invention relates to a method and a device for noninvasive blood pressure measurement by means of a pressure cuff and a sensor, the output signal S of which depends on the blood pressure Pi to be measured and on the cuff pressure Pm, characterized in that first in a reference phase the cuff pressure Pm is varied and the maximum output signals Smax of the sensor occurring at the different cuff pressures Pm at the systolic blood pressure value Ps; the dependence of the maximum output signal Smax of the signal occurring at the systolic blood pressure value Ps on the transmural pressure Pt is determined, from which the curve of a reference phase quotient qR is determined as a function of the transmural pressure Pt and saved; then in a measurement phase, signal values SpM1 and SpM2 are determined during one pulse beat at two different cuff pressures pM1 and pM2, and a measurement phase quotient qM is formed from the two signal values; and the blood pressure Pi is determined by a comparison of the measurement phase quotient qM with the stored reference phase quotient qR.

In one embodiment, the invention relates to a method for noninvasive blood pressure measurement by means of two pressure cuffs, each with one sensor, the output signal of which is a function of the blood pressure to be measured and of the cuff pressure, characterized in that first in a reference the cuff pressure is varied and the maximum output signals of the sensor occurring at the different cuff pressures at the systolic blood pressure value are detected; the systolic blood pressure value occurring during the reference phase is known or determined; the dependence of the maximum output signal of the sensor, occurring at the systolic blood pressure value, on the transmural pressure is determined by using the known or determined systolic blood pressure value and the relationship Pt=Pi−Pm, according to which the transmural pressure Pt prevailing in the vascular wall is equal to the difference between the blood pressure Pi and the cuff pressure Pm, and then from this the curve of a reference phase quotient qR is determined as a function of the transmural pressure Pt as being

qR(Pt)=Smax(Ps−Pm)/Smax(Ps−Pm−dPm)

and is saved, the differential cuff pressure dPm being less than 50 mmHg; then in a measurement phase with a control unit, two signal values are determined in automated fashion at two different cuff pressures during one pulse beat, and a measurement phase quotient is formed from these two signal values; and by comparing the measurement phase quotient with the saved reference phase quotient, the blood pressure is determined. The control unit may also alternatively or additionally automatically control the reference phase.

The measurement phase may persist for a long time, in particular as long as the patient's vascular condition is essentially unchanged. The reference phase can be repeated automatically as needed or at regular intervals of time, so that the burden for the patient is minor. In the reference phase the cuff pressure may be varied over a wide range, for example, from 0 to 150 mmHg. For measurement of the blood pressure, i.e., in the measurement phase, the cuff pressure may always remain below the diastolic pressure. Measurements may therefore be performed repeatedly or continuously. This also yields the possibility of achieving a high precision and eliminating artifacts.

The pulse stroke is determined by comparing its effect on the signal level with the change in the transmural pressure by a defined and known amount within a pulse beat. The tension level of the vessel beneath the cuff is characterized by the ratio of two signal values, which are measured at two cuff pressures that differ by a certain value. The signal value may be represented as a product and as an integral. This comparison allows a determination of the systolic and diastolic blood pressure values.

The method according to the invention makes use of the known physiological and physical principles but without repeated constriction of the vessels. For the individual measurement it offers a possibility for deriving a second pressure value, without empirical assumptions from measurements, in addition to the systolic pressure value.

The invention shows how the diastolic blood pressure value can be determined merely from objective measured values. Even with longer monitoring of the blood pressure, for example, in a long-term monitoring of the patient for several days or even weeks, the cuff pressure remains below the diastolic pressure, which is very pleasant for the patient. During the monitoring, the curve of the blood pressure over time can be recorded approximately and output or displayed. The device according to the invention may have an output interface and/or a display device for this purpose.

The following terms and abbreviations are used here:
Ps systolic blood pressure
Pm cuff pressure
Pd diastolic blood pressure
Pt transmural pressure, pressure in the vascular wall
Pi pressure in vessel
Pb(t) internal vascular pressure
Ps-Pd referred to as the pulse stroke; this is the greatest pressure difference in the vessel within one pulse beat
φ(Pm) measured signal value as a function of Pm
S(Pt) signal value caused by the pulse stroke as function of Pt
Smax maximum signal value within a pulse beat
W(Pt) or F(Pt) the curve describing the vascular expansion status
u(Pt) the differential of W(Pt), dW(Pt)/dPt
f(Pt) the differential of F(Pt), dF(Pt)/dPt

Pressures are given in Torr=mmHg

The device according to the invention may have a pressure cuff, an air pump, a pressure meter and a discharge value as is the case with conventional blood pressure measurement systems according to Riva-Rocci or Korotkow.

Furthermore, the device has a control unit, in particular a control computer with which the measurement sequence can be automated. The control unit may have a memory in which a control program of the control unit and/or the measured and determined and/or calculated values can be saved. For the embodiment with optical signal generation, a photoelectric sensor with a transmitter, a receiver and an electronic unit with a signal amplifier are required.

A pressure cuff for the upper arm, the wrist or a finger, for example, may be used for the invention. With each type of cuff, the pressure pulse occurring in the cuff due to the increase in the vascular volume because of the pulse stroke may be analyzed as a signal. A second type of signal generation is possible using a finger cuff, for example: a photoelectric sensor with a transmitter and receiver is placed beneath the cuff and used to pass a beam of light through the finger. The pulse stroke causes an increased absorption of the light and the inverted value of this measurement corresponds to the variable referred to as a signal in this description. With a pressure measurement device, the absolute pressure in the cuff and the pressure pulses may also be measured.

The blood pressure measurement is performed in two steps. In a reference phase a reference curve is measured on the patient and defined, and may be used as a reference for this patient and this measurement site as long as the elasticity of the vascular system does not change significantly, i.e., for a few weeks, for example. However, the measurement of the reference curve is not very complicated and therefore may be repeated frequently. In the measurement phase the blood pressure is determined and monitored with the help of this reference curve. The blood pressure curve may be performed continuously over a longer period of time, and the blood pressure may be recorded in the curve of the pulse beat, wherein the cuff pressure never exceeds the diastolic blood pressure.

To record the reference curve, the cuff is acted upon by a pressure which is greater than the diastolic pressure. The pressure is therefore increased until a signal is no longer measureable because the vessels have collapsed completely. Then the cuff pressure is reduced slowly. The pressure at which the first signals are measurable may be recorded as the systolic blood pressure Pso in the optical measurement system. In measurement of the pressure pulses this is not the case because, even with completely collapsed vessels on the proximal end of the cuff, signals are still generated. However, since the accuracy of this value enters into all subsequent measurements, the blood pressure can also be measured promptly by any other method, even invasively, to increase the accuracy. Then, as a rule, the pulse stroke is preferably measured with the method according to the invention to be described below in addition to measurement of the systolic blood pressure.

During the reduction to Pm=0, the signal may be recorded as a function of the cuff pressure as S=φ(Pm) and saved in tabular form. Use of this curve as a reference is facilitated if the curve values for integral pressure values are determined in mmHg by interpolation methods. How the interpolation is best to be performed will depend to a great extent on the number of interpolation points in the plot of S=φ(Pm). For example, the measurement points may be connected by a combination of two polynomials and interpolated by using a cubic spline.

Additional advantages, features and details of the invention are derived from the dependent claims as well as the following description in which several exemplary embodiments of the invention are described in detail with reference to the drawings. The features mentioned in the claims and the description may each be essential to the invention individually or in any combination.

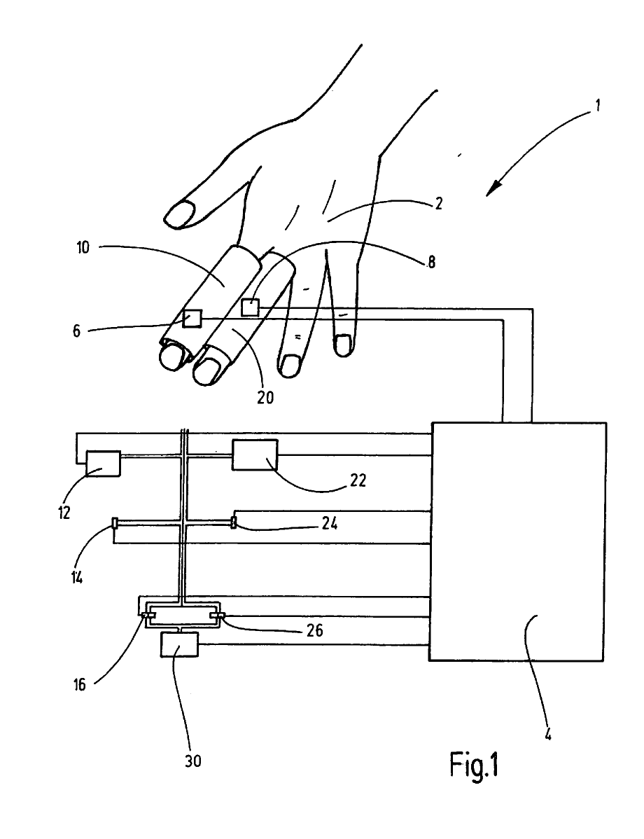

FIG. 1 shows an exemplary embodiment of an device according to the invention for noninvasive blood pressure measurement,

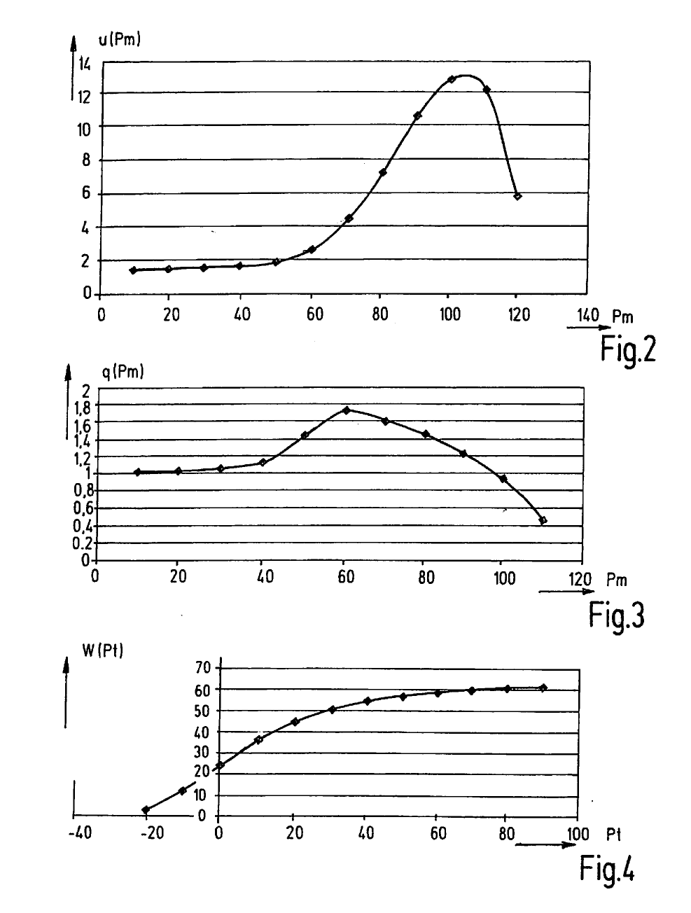

FIG. 2 shows the curve u(Pm) determined by interpolation.

FIG. 3 shows the curve of the quotient q(Pm),

FIG. 4 shows the curve W(Pt) described by the distension state of the vessel under the cuff,

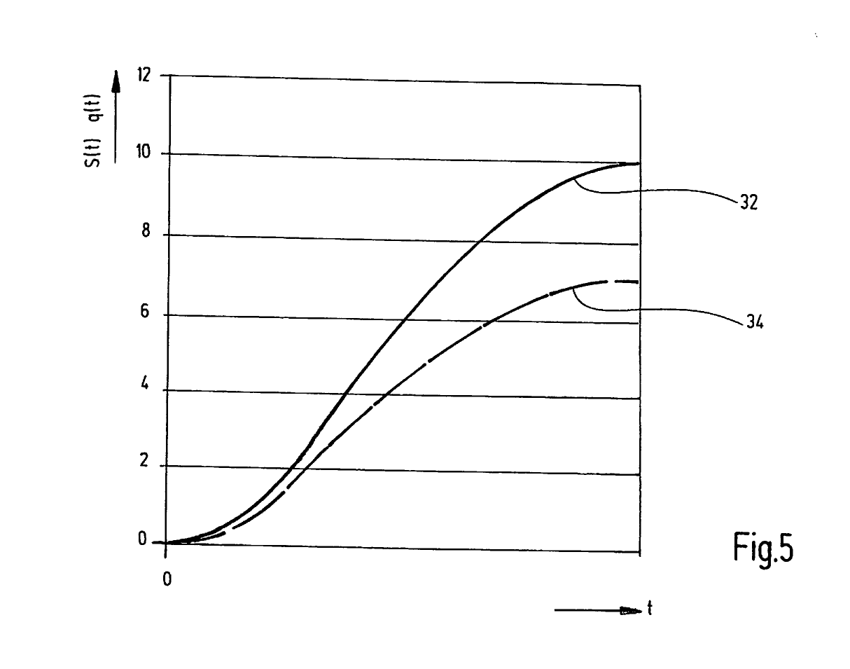

FIG. 5 shows the curve of the measurement phase quotient qM(t) and

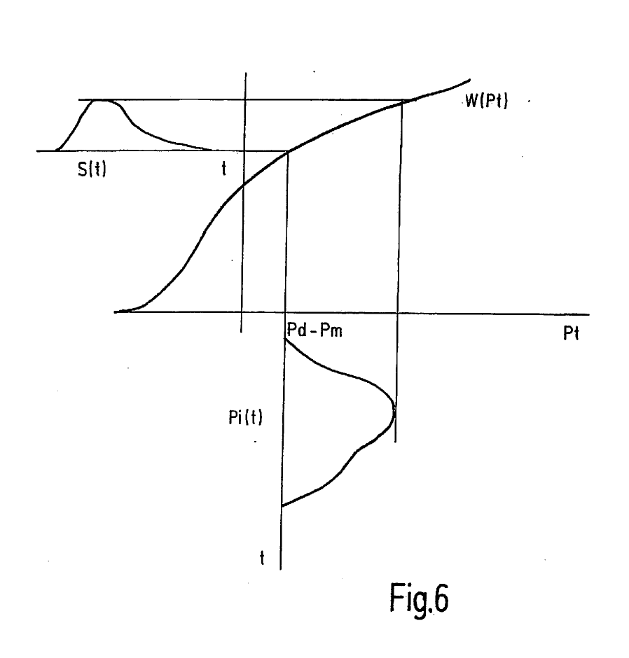

FIG. 6 shows an example of a conversion of the pulse curve S(t) into the blood pressure curve Pi(t).

FIG. 1 shows one exemplary embodiment of a device 1 according to the invention for noninvasive blood pressure measurement. The device 1 has two cuffs 10, 20, each of which is applied to an extremity, namely each being applied to one finger of a hand 2 in the exemplary embodiment. The filling pressure of the cuffs 10, 20 is measured independently by a pressure transducer 12, 22. The two cuffs 10, 20 can each be cut off or filled with air through a valve 14, 24 and can be connected to a pump 30, in particular an air pump, by means of another valve 16, 26.

The valves 14, 24 and additional valves 16, 26, like the pump 30, are connected to a control unit 4, which is also connected to the pressure transducer 12, 22 and to the sensors 6, 8 of the two cuffs 10, 20. One difference in the exemplary embodiment here in comparison with the known devices is that measurement signals are picked up at two locations on the hand 2, simultaneously and independently of one another.

The pressure fluctuations caused by the pulse beat can be detected as measurement signals in the sensors 6, 8 and used to determine the blood pressure. However, an alternative embodiment uses optical signals and uses photoelectric sensors, which are mounted on the cuff 10, 20, for example, instead of pressure sensors. Use of optical sensors 6, 8 also permits a simple combination of the present method according to the invention for noninvasive blood pressure measurement with the optical measurement of the oxygen saturation of the blood.

First, the systolic blood pressure Ps is measured on the patient and data is recorded for a reference table. With the same cuff pressure in both cuffs, a signal amplifier is set so that the output signals of the two sensors 6, 8 are the same. This step may be repeated to increase the accuracy of the measurement method, in particular being repeated regularly and in an automated fashion. For example, an electronic measurement unit disposed in the control unit 4 may perform the setting of the gain automatically as soon as a measurement is performed at the same pressure in both cuffs.

Next the cuff pressure is increased in both cuffs 10, 20 until both sensor signals almost disappear or are below a predefined minimum value. The measured pressure here is recorded as the systolic blood pressure Ps of the patient.

Then the pressure in the first cuff 10 is lowered by a value dPm, next a pulse curve is recorded for both fingers and then the pressure in the second cuff 20 is lowered to the same value and a control measurement may be performed. This cycle is repeated until the cuff pressure Pm has reached a lower predefined value, of 10 mmHg, for example.

Then the differential pressure value dPm may amount to any value but it should not be too small or too large and in particular should be more than 5 mmHg and less than 50 mmHg, preferably more than 5 mmHg and less than 20 mmHg. This value is 10 mmHg in the exemplary embodiment.

FIG. 2 shows the curve u(Pm) of the maximum signal value Smax, determined by interpolation, as a function of the cuff pressure Pm. In the exemplary embodiment shown here, the systolic blood pressure value Ps, which is known during the reference phase or is determined by some other method, amounts to 130 mmHg.

FIG. 3 shows the curve of the quotient q(Pm)=Smax(Pm+dPm)/Smax(Pm) which is derived from the measured values. Since the maximum signal value Smax always occurs at the systolic blood pressure value Ps, the curve u(Pm) in FIG. 3 can be scaled up from the cuff pressure Pm to the transmural pressure Pt=Pi−Pm=Ps−Pm.

FIG. 4 shows the curve W(Pt), which describes the distension condition of the vessel beneath the cuff 10, 20 and which can be determined by numerical integration of the curve u(Pt).

For the additional measurements, the curves q(Pt) and W(Pt) are approximated using suitable mathematical means, for example, a parabolic interpolation, so that sufficiently accurate estimates are available for all intermediate values. The curve of q(Pt) is supplied as a table. W(Pt) is required for conversion of pulse curves into blood pressure curves, not for determination of the blood pressure values. All this data can be used for the patient being tested as long as the elasticity of the patient's blood vessel does not change significantly, which is usually the case for several weeks or months.

For long-term monitoring of blood pressure, the first cuff 10 is subjected to a pressure between 20 mmHg and 60 mmHg, for example, in the measurement phase, whereas the second cuff 20 is subjected to cuff pressure which is higher or lower than the former by a differential value dPm. Next the pulse curve is recorded on both cuffs 10, 20 and/or fingers, i.e., the curve of the sensor signal S(t) over time. Since the following equation holds for both pulse curves

S(t)=(Pi(t)−Pd)·u(Pt(t))

q values are obtained as measurement phase quotient qM(t) by dividing the ordinates at each point in the pulse curve as shown in FIG. 5.

At the peak of the two pulse curves 32, 34, a q value is obtained for the systolic blood pressure value Ps. The pulse curves 32, 34 start at the diastolic blood pressure value Pd, namely at the time t=0 in the diagram in FIG. 5, at the time when the formation of a q value is impossible. However, a value can be extrapolated for t=0 by using the q values around t=0. Since the cuff pressure Pm is known, the diastolic blood pressure value Pd can be obtained from the table q(Pt) using the value thus obtained for q. The slope of the extrapolation curve indicates whether the q value for Pd occurs before or after the maximum of q(Pt). In this way, both the systolic blood pressure value Ps and the diastolic blood pressure value Pd can be measured without having to increase the cuff pressure.

The values obtained by division of the ordinates form the curve q(Pt) approximately, but they converge exactly at the q value for the diastolic blood pressure Pd at t=0. The execution of the extrapolation depends on the scattering in the measured data at a point in time t=0. The pulse curves 32, 34 up to the maximum value are sufficient for determination of the blood pressure values. If the blood pressure curve is to be diagrammed completely, the pulse curves 32, 34 must be recorded completely.

FIG. 6 shows an example of conversion of the pulse curve S(t) into the blood pressure curve Pi(t). In addition to the curve W(Pt), the prevailing diastolic blood pressure Pd must be known at each pulse beat for the recording.

1. A method for noninvasive blood pressure measurement by means of two pressure cuffs (10, 20), each having a sensor (6, 8), the output signal (S) of which depends on the blood pressure (Pi) to be measured and on the cuff pressure (Pm), characterized in that
first, in a reference phase, the cuff pressure, (Pm) of at least one pressure cuff (10, 20) is varied, and the maximum output signals (Smax) of the sensor occurring at the systolic blood pressure (Ps) are detected at the different cuff pressures (Pm),
the systolic blood pressure value (Ps) occurring during the reference phase is known or is determined,
when using the known or determined systolic blood pressure value (Ps) and the relationship Pt=Pi−Pm, according to which the transmural pressure (Pt) prevailing in the vascular wall is equal to the difference between the blood pressure (Pi) and the cuff pressure (Pm), the dependence of the maximum output signal (Smax) of the sensor (6, 8), occurring at the systolic blood pressure (Ps), on the transmural pressure (Pt) is determined, and the curve of the reference phase quotient (qR) is determined as a function of transmural pressure (Pt) and saved as qR(Pt)=Smax(Ps−Pm)/Smax(Ps−Pm−dPm), wherein the differential cuff pressure (dPm) is less than 50 mmHg,
then, in a measurement phase during a pulse beat, two signal values (SpM1; SpM2) are determined at two different cuff pressures (pM1; pM2) by means of a control unit (4), and a measurement phase quotient (qM) is formed from these two signal values (SpM1; SpM2), and, by comparing the measurement phase quotient (qM) with the saved reference phase quotient (qR), the blood pressure (Pi) is determined.

2. The method according claim 1, characterized in that
a pulse curve, which has a first peak (Smax1) at the time of occurrence of the systolic blood pressure (Ps), is recorded with a first cuff at a first cuff pressure (Pm1),
using another cuff, a pulse curve, which has a second peak (Smax2) at the point in time of occurrence of the systolic blood pressure (Ps), is recorded at the same time at a second cuff pressure (Pm2), which is different from the first cuff pressure (Pm1) by the differential cuff pressure (dPm),
a measurement phase quotient (qM) is formed from the first and second maximums (Smax1, Smax2),
the transmural pressure (Pt) prevailing at the point in time of occurrence of the two peaks (Smax1, Smax2), and thus at the point in time of occurrence of the systolic blood pressure (Ps), is determined by comparison of the measurement phase quotient (qM) with the saved reference phase quotient (qR) and then with the help of the known cuff pressure (Pm) the systolic blood pressure (Ps) is determined from this according to Pi=Ps=Pt+Pm.

3. The method according claim 1, characterized in that the diastolic blood pressure value (Pd) is calculated by extrapolation, in particular by extrapolation of the measurement phase quotient (qM) which occur at a near the minimum measured output signals (Smin1, Smin2).

4. A device (1) for noninvasive blood pressure measurement using two pressure cuffs (10, 20), each with a sensor (6, 8), the output signal (S) of which depends on the blood pressure (Pi) to be measured and on the cuff pressure (Pm), wherein the device (1) also has a control unit (4), characterized in that by means of the control unit (4), the cuff pressure (Pm) of at least one pressure cuff (10, 20) can be varied at first in a reference phase, and the maximum output signals (Smax) of the sensor occurring at the systolic blood pressure value (Ps) at each of the different cuff pressures (pM) can be detected; the systolic blood pressure value (Ps) occurring during the reference phase is known or is determined; using the known or determined systolic blood pressure value (Ps), and the equation Pt=Pi−Pm, according to which the transmural pressure (Pt) prevailing in the vascular wall is equal to the difference between the blood pressure (Pi) and the cuff pressure (Pm), the relationship of the maximum output signal Smax of the sensor to the transmural pressure (Pt) occurring with the systolic blood pressure value (Ps) can be determined, and from that, the curve of a reference phase quotient (qR) can be determined as a function of the transmural pressure (Pt) as qR(Pt)=Smax(Ps−Pm)/Smax(Ps−Pm−dPm) and saved, wherein the differential cuff pressure (dPm) is less than 50 mmHg; then in a measurement phase during a pulse beat, two signal values (SpM1; SpM2) can be determined during one pulse beat at two different cuff pressures (pM1; pM2) and a measurement phase quotient (qM) can be formed from these two signal values (SpM1; SpM2) and the blood pressure (Pi) can be determined by a comparison of the measurement phase quotient (qM) with the saved reference phase quotient (qR).

5. The device (1) according to claim 4, characterized in that the device (1) has two cuffs (10, 20) and two sensors (6, 8) by means of which the pulse curve can be detected in a measurement phase at two different cuff pressures (Pm1, Pm2) which differ by a differential pressure dPm.

6. The device (1) according to claim 4, characterized in that the sensor(s) (6, 8) of the device (1) is/are optical sensors.
